(19) United States
(12) Patent Application Publication
(10) Pub. No.: US 2013/0190820 A1
(43) Pub. Date: Jul. 25, 2013

(54) SPINOUS PROCESS IMPLANT WITH GEAR TEETH

(71) Applicants: Daniel E. Siegfried, Memphis, TN (US); Bradley E. Steele, Germantown, TN (US); Jeetendra S. Bharadwaj, Hyderabad (IN)

(72) Inventors: Daniel E. Siegfried, Memphis, TN (US); Bradley E. Steele, Germantown, TN (US); Jeetendra S. Bharadwaj, Hyderabad (IN)

(73) Assignee: Warsaw Orthopedic, Inc., Warsaw, IN (US)

(21) Appl. No.: 13/357,662

(22) Filed: Jan. 25, 2012

(51) Int. Cl. A61B 17/70; A61B 17/88

(52) U.S. Cl. 606/248; 606/279

(57) ABSTRACT

Devices and methods for spinous process implants with two plates that are connected together by a post, with each plate configured to be positioned on outer lateral sides of spinous processes with the post extending through the interspinous space. The second of the two plates includes a bore that receives the post, and that plate is movable along the length of the post to accommodate different anatomies, and selectively lockable in position. The post includes external gear teeth. A tool with a drive gear is used to releasably gearingly engage the gear teeth of the post and drive the second plate toward the first plate. When the plates are in their desired locations, the second plate is locked in position and the surgical tool may be removed.

### BACKGROUND

The present invention generally relates to devices and methods for stabilizing vertebral members, and more particularly, to spinal implants that mount onto the spinous processes.

Vertebral members typically comprise a vertebral body, pedicles, laminae, and processes. The processes are projections that serve as connection points for the ligaments and tendons, and typically include the articular processes, transverse processes, and the spinous process. Intervertebral discs are located between adjacent vertebral bodies to permit flexion, extension, lateral bending, and rotation.

Various conditions may lead to damage of the intervertebral discs and/or the vertebral members. The damage may result from a variety of causes including a specific event such as trauma, a degenerative condition, a tumor, or infection. Damage to the intervertebral discs and vertebral members can lead to pain, neurological deficit, and/or loss of motion. One manner of correcting the damage involves mounting of a spinal implant onto the spinous processes, typically in association with a fixation process such as anterior lumbar interbody fusion (ALIF), posterior lumbar interbody fusion (PLIF), intertransverse lumbar interbody fusion (ILIF), and the like. See, for example, the spinal implant sold under the trade name CD HORIZON SPIRE™ by Medtronic Spinal and Biologics of Memphis, Tenn., and the devices described in U.S. Pat. Nos. 7,048,736 and 7,727,233. While these devices provide some solutions, they may not be ideal for some situations. As such, there remains a need for alternative spinal implants and related methods.

### SUMMARY

The present application is directed to implants for attaching to spinous processes and/or methods of attaching an implant to spinous processes. The implants typically includes two plates that are connected together by a post. The implant is configured for each plate to be positioned on outer lateral sides of spinous processes with the post extending through the interspinous space. The second of the two plates includes a bore that receives the post, and that plate is movable along the length of the post to accommodate different anatomies such as for relatively wide or thin spinous processes, and selectively lockable in position. The post includes external gear teeth. A tool with a drive gear is used to gearingly engage the gear teeth of the post and drive the second plate toward the first plate. When the plates are in their desired locations, the second plate is locked in position and the surgical tool subsequently removed. The use of the gear teeth on the post facilitates insertion of the spinal implant, for example by reducing the number of surgical tools required to implant the device.

In some embodiments, a method of attaching an implant to spinous processes is provided. The method includes positioning a first plate on a first lateral side of the spinous processes, positioning a second plate on a second lateral side of the spinous processes in spaced relation to the first plate, and positioning a post that extends outward from the first plate through an interspinous space formed between the spinous processes and through a bore in the second plate. The post extends along a post longitudinal axis from a proximal section attached to the first plate to a distal section positioned distally from the first plate. The post distal section has a plurality of external gear teeth thereon disposed in series at increasing spacing from the first plate. The method includes removably coupling a tool to the gear teeth. Thereafter, the second plate is moved along the post axis toward the first plate by engaging successive gear teeth of the post with the tool so that the tool pushes the second plate toward the first plate. Thereafter, the second plate is locked relative to the post.

The method may further comprise thereafter decoupling the tool from the gear teeth. The method may comprise engaging a fastener with the second plate prior to the locking, with the locking the second plate comprising advancing the fastener toward the post axis. The method may comprise pivoting the post relative to the first plate prior to the locking. The method is advantageously such that moving the second plate toward the first plate comprises causing protrusions on the first and second plates to bite into opposing lateral sides of adjacent spinous processes. The tool may comprise a ratcheting mechanism, and engaging successive gear teeth of the post with the tool may comprise rotating a drive gear of the tool in a first direction and inhibiting rotation of the drive gear in a second opposite direction via the ratcheting mechanism. The removably coupling the tool the gear teeth may comprise, after the positioning the first plate, the positioning the second plate, and the positioning the post, moving the tool in an anterior direction and into engagement with the implant; and mating a drive gear of the tool with one or more of the gear teeth of the post. The method may be such that engaging successive gear teeth of the post with the tool comprises rotating a drive gear of the tool in a first direction about a gear axis disposed normal to the post axis while the drive gear is in contact with the gear teeth of the post.

In some embodiments, the first plate has a longitudinal axis, and the gear teeth point in a direction generally parallel to the first plate longitudinal axis and generally normal to the post axis, and engaging the gear teeth with the tool comprises rotating a drive gear of the tool about an axis that is generally normal to the first plate longitudinal axis and generally normal to the post axis. In some embodiments, the first plate has a longitudinal axis, and the gear teeth point in a direction generally normal to the first plate longitudinal axis and generally normal to the post axis, and engaging the gear teeth with the tool comprises rotating a drive gear of the tool, while engaged with the gear teeth, about an axis that is generally parallel to the first plate longitudinal axis and generally normal to the post axis.

In some embodiments, a spinal implant is provided. The implant includes a first plate sized to extend along a first lateral side of the spinous processes and a second plate sized to extend along a second lateral side of the spinous processes. The second plate includes a first bore that extends through the second plate between a medial surface that faces towards the first plate and an opposing outer surface. An elongated post that extends along a post longitudinal axis from a proximal section attached to the first plate to a distal section positioned distally from the first plate. The distal section has a plurality of external gear teeth thereon disposed in series at increasing spacing from the first plate. The first bore is sized relative to the post for the second plate to be infinitely movable along the post and selectively locked in position therealong. A fastener is engageable with both the second wing and the post to selectively lock the second wing relative to the post. The second plate may further comprise a fastener bore intersecting the first bore, with the fastener disposed in the bore. The fastener may be a setscrew. The fastener may engage the post from a direction orthogonal to the post axis. The gear teeth may be disposed in series aligned normal to the post axis. The first and second plates may advantageously each comprise a plurality of protrusions extending toward each other, with the protrusions configured to bite into the spinous processes. The gear teeth may extend not more than partially circumferentially around the post axis. At least some of the gear teeth may be disposed in the first bore. The first plate may have a longitudinal axis and the gear teeth may point in a direction generally parallel to the first plate longitudinal axis and generally normal to the post axis. Alternatively, the first plate may have a longitudinal axis and the gear teeth point in a direction generally normal to the first plate longitudinal axis and generally normal to the post axis.

The various aspects of the various embodiments may be used alone or in any combination, as is desired.

### BRIEF DESCRIPTION OF THE DRAWINGS

FIG. 1 shows a spinal implant according to one embodiment mounted to a spinal column.

FIG. 2 shows a perspective view of another embodiment of a spinal implant.

FIG. 3 shows a different perspective view of the implant of FIG. 2 with a surgical tool attached.

FIG. 4 shows the surgical tool of FIG. 3 in more detail.

FIG. 5 shows a drive gear of the surgical tool of FIG. 4 engaged with gear teeth of the implant of FIG. 2, with other portions of the tool removed for clarity.

FIG. 6 shows a perspective view of another embodiment of a spinal implant.

FIG. 7 shows a top view of the implant of FIG. 6.

FIG. 8 shows the implant of FIG. 6 with a surgical tool attached.

FIG. 9 shows a side view of the surgical tool of FIG. 8.

### DETAILED DESCRIPTION

In one embodiment, the present application is directed to a spinal implant with two plates that are connected together by a post. The implant is configured for each plate to be positioned on outer lateral sides of spinous processes with the post extending through the interspinous space. The second of the two plates includes a bore that receives the post, and that plate is movable along the length of the post to accommodate different anatomies such as for relatively wide or thin spinous processes, and selectively lockable in position. The post includes external gear teeth. A tool with a drive gear is used to gearingly engage the gear teeth of the post and drive the second plate toward the first plate. When the plates are in their desired locations, the second plate is locked in position and the surgical tool subsequently removed. The use of the gear teeth on the post facilitates insertion of the spinal implant, for example by reducing the number of surgical tools required to implant the device.

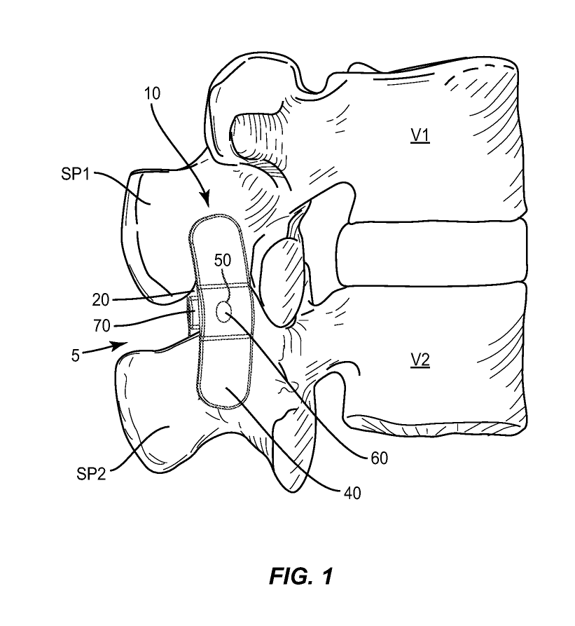

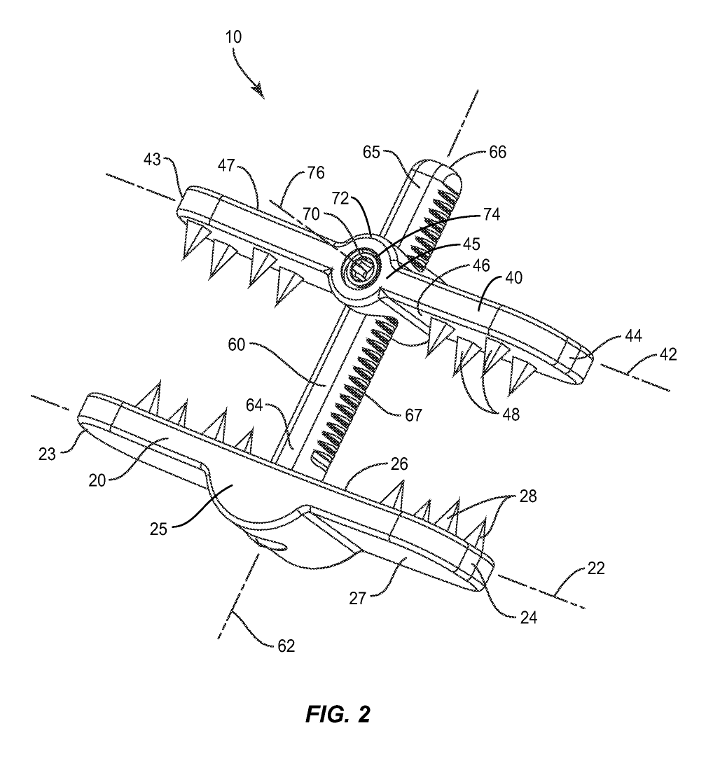

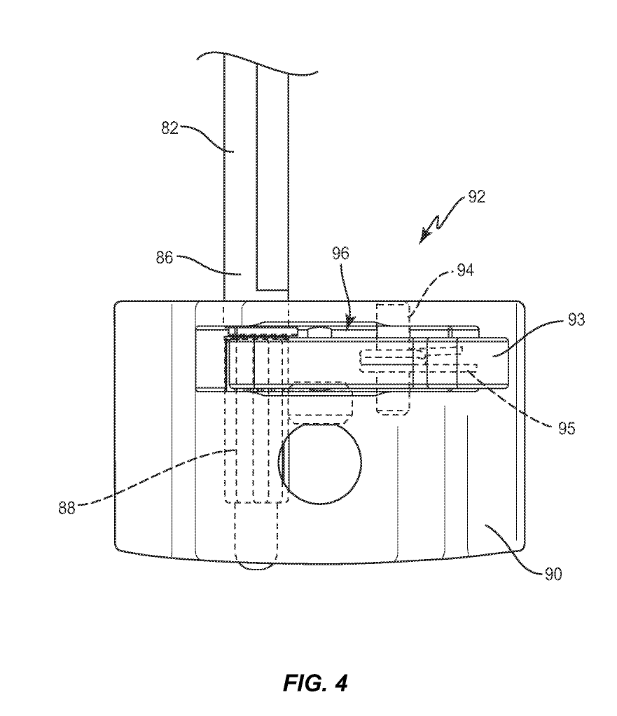

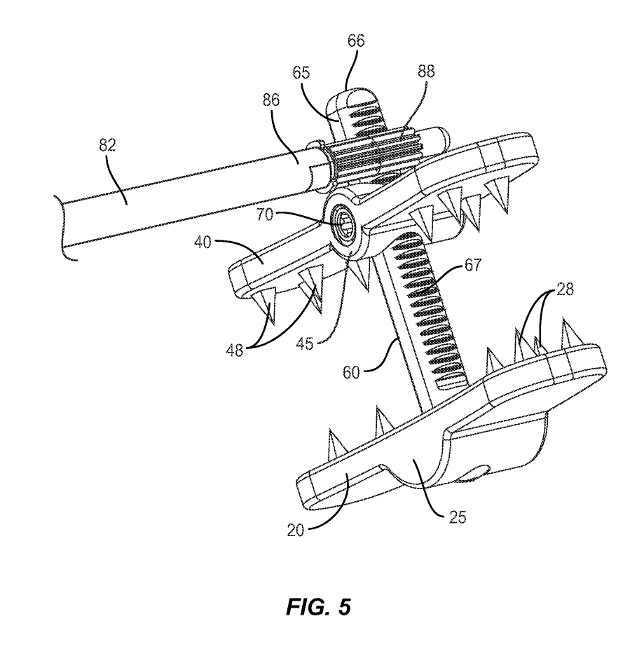

Referring to FIG. 1, a spinal implant according to one embodiment and generally designated 10 is shown clampingly mounted to the spinous process SP1 of a superior vertebra V1 and a spinous process SP2 of an inferior vertebra V2. A portion of the implant 10, in particular post 60, extends transversely through the interspinous space 5 between the two spinous processes SP1,SP2. The implant 10, shown more clearly in FIGS. 2-5, includes a first plate 20, a second plate 40, an interconnecting post 60, a fastener 70, and a gear nut 80.

Referring to FIGS. 2-3 and 5, the first plate 20 may be elongate along an associated longitudinal axis 22, with a superior end section 23, an inferior end section 24, and an intermediate section 25. If desired, the superior end section 23 and inferior end section 24 may be shifted in an anterior direction or a posterior direction so that the first plate has a somewhat Z-shape, although this is optional and the first plate 20 may be generally continuously gently curved, rectilinear, or any other suitable shape as is desired. The first plate 20 has a length sufficient to vertically span the interspinous gap 5 (interspinous space) between adjacent spinous processes while substantially overlapping the spinous processes SP1,SP2. The first plate 20 has a medial face 26 and an opposite lateral face 27. The medial face 26 includes a plurality of protrusions 28 that extend medially for biting into the corresponding spinous process SP1,SP2. Advantageously, the protrusions take the form of a plurality of sharp teeth. The teeth 28 may advantageously be disposed in two groups, one on the superior end section 23 and one on the inferior end section 24, with the intermediate section 25 being free of such teeth 28. The lateral face 27 may have suitable features, such as recesses or the like, for cooperating with installation and manipulation instrumentation. The tips of superior end 23 and inferior end 24 are advantageously generally rounded so as to minimize damage to surrounding tissue and for ease of installation.

The second plate 40 may be substantially similar to the first plate 20. For example, the second plate 40 may be elongate along an associated longitudinal axis 42, with a superior end section 43, an inferior end section 44, and an intermediate section 45. If desired, the second plate 40 may be shaped similar to the first plate 20, or may be any other suitable shape as is desired. The second plate 40 advantageously has a length sufficient to vertically span the interspinous gap 5 while substantially overlapping the spinous processes SP1,SP2. The second plate 40 has a medial face 46 and an opposite lateral face 47, with the medial face 46 facing the medial face 26 of the first plate 20. The medial face 46 includes a plurality of protrusions 48 similar to teeth 28 for biting into the spinous processes SP1,SP2. The lateral face 47 may have suitable features, such as recesses or the like, for cooperating with installation and manipulation instrumentation. The tips of superior end section 43 and inferior end section 44 are advantageously generally rounded so as to minimize damage to surrounding tissue and for ease of installation. The intermediate section 45 may have a suitable boss 72 thereon, with a hole 74 having centerline 76 for receiving the fastener 70, as discussed below. The intermediate section 45 of the second plate 40 includes a bore 50 that extends from medial face 46 to lateral face 47, through the intermediate section 45. The bore 50 is sized to receive post 60, and therefore has a cross-section at least as large, and advantageously slightly larger than, the corresponding portion of post 60.

The post 60 may take the form of a round shaft that extends along a post longitudinal axis 62 from a post proximal section 64 proximate the first plate 20 to a post distal section 65 proximate the second plate 40. The post 60 has a length sufficient to extend laterally across the interspinous gap 5 and through the bore 50 of second plate 40. In some embodiments, the post proximal section 64 is mounted to the first plate 20 by any suitable means, such as welding or the like. Alternatively, the post 60 may be integrally formed with the first plate 20. Still further, the post 60 may be pivotally attached to the first plate 20. For example, the post 60 may be pivotally attached either for monoaxial or polyaxial movement relative to the first plate 20 about one or more pivot axes, such as about a pivot axis perpendicular to the post axis 62 and parallel to medial face 26. Examples of suitable pivoting structures are shown in U.S. Pat. Nos. 7,048,736 and/or 7,727,233. The post 60 may include flats or other features (not shown) for engaging with the fastener 70. The post 60 may advantageously be rigid and generally solid. The distal section 65 terminates at a distal tip 66. The post distal section 65 advantageously has a cylindrical cross-sectional shape and includes external gear teeth 67. Gear teeth 67 extends proximally a significant distance, and may extend all the way to or into the proximal section 64. Gear teeth are arranged in series so as to form a gear rack, with adjacent teeth being a varying distance from medial face 26 of first plate 20. The tips of gear teeth 67 point in a direction that is normal to both longitudinal axis 22 and post axis 62. The gear teeth 67 may be disposed so that the series of gear teeth 67 is aligned parallel to the medial face 48, or at any suitable transverse angle such as at up to about 30°. As can be appreciated, the gear teeth 67 may extend circumferentially completely around post axis 62, but advantageously extend circumferentially around approximately one quarter thereof.

The fastener or locking member 70 may take the form of a simple setscrew, optionally with tapered tip, that is sized to threadably engage hole 74 in second plate 40. When tightened, the locking member 70 presses against a portion of gear nut 80 to lock the relative distance between the plates 20,40. Of course, other forms of fasteners, such as concentrically barbed posts, quarter-turn fasteners, and the like, may alternatively be used.

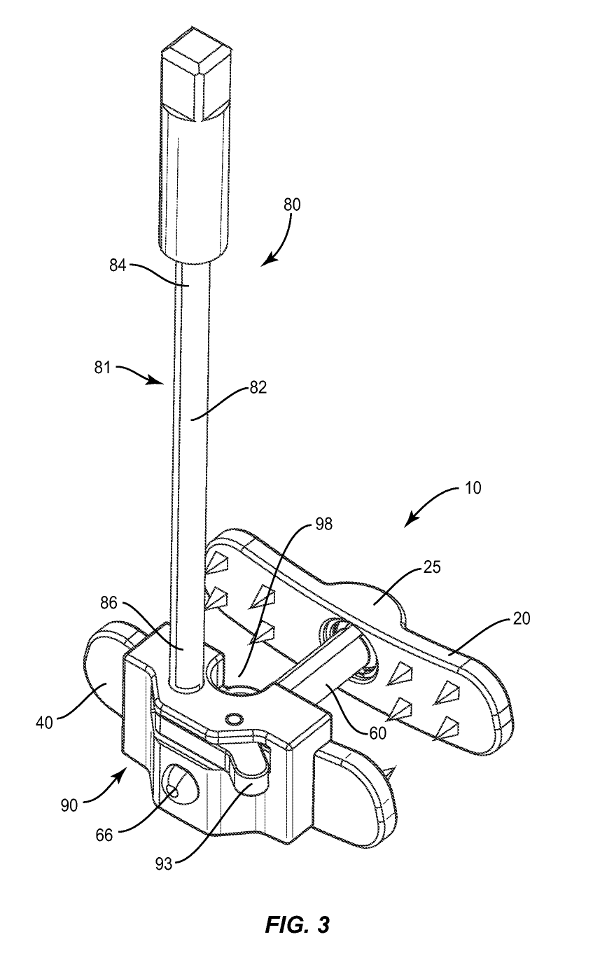

A surgical tool 80 is used to gearingly drive the second plate 40 toward the first plate 20 via engagement with gear teeth 67. In one embodiment shown in FIGS. 3-5, the surgical tool 80 includes a driver 81 and a base unit 90. The driver 81 advantageously includes an elongate shaft 82 having a drive gear section 88 (or simply drive gear) on the distal section 86 thereof. The proximal section 84 of the shaft 82 is suitably configured to receive a rotational drive input, such as an end having a generally square cross-section as shown in FIG. 3. The drive gear 88 is configured to matingly engage with gear teeth 67. The base unit 90 includes a housing 91 that receives both post 60 and driver 81, and is configured to abut against at least lateral face 47 of second plate 40. The base unit 90 further advantageously includes a ratcheting mechanism 92. The ratcheting mechanism 92 may include a pawl 93 pivotally mounted to the base unit 90 via a pivot pin 94, and biased toward an engaging position (with driver gear 88) by a spring 95. The pawl 93 is pivotally mounted in a cavity 96 that opens onto the driver 81. The pawl 93 engages the drive gear 88 and allows the drive gear 88 to rotate in one direction, but inhibits rotation in the other direction. The driver 81 is mounted to the base unit 90 and extends into a passage that opens onto the post 60 when the post 60 is engaged by the surgical tool 80. The driver 81 may be mated to the base unit 90 in any convenient fashion that allows the driver to rotate, such as with a spring clip or the like. The base unit 90 includes a notch 98 or other recess that allows access to the fastener 70, and/or allows the fastener 70 to be inserted into hole 74, when the surgical tool 80 is mated to the second plate 40.

In use, the device 10 can be implanted for posterior spinal stabilization as a stand-alone procedure or in conjunction with other procedures. The device 10 can be positioned through a small posterior incision in the patient of sufficient size to admit the device and instrumentation. Following the incision, muscle is moved aside if and as needed for placement of the device 10. The spinous processes SP1,SP2 are optionally distracted using suitable instrumentation known in the art, and the first plate 20 is implanted such that the superior end section 23 extends on a first lateral side of spinous process SP1, inferior end section 24 extends on the first side of spinous process SP2, and post 60 extends through the interspinous space 5 generally normal to the sagittal plane defined by the spinous processes SP1,SP2. This implantation may involve pivoting of the post 60 in some embodiments. With the first plate 20 in position, the distal tip 66 of post 60 extends laterally beyond the spinous processes SP1,SP2 on the lateral side opposite first plate 20. The second plate 40 may then be added by inserting post 60 into bore 50, and sliding the second plate 40 on post 60 slightly toward the first plate 20. As the second plate 40 is slid toward the first plate 20, the post tip 66 becomes positioned distally beyond the lateral face 47 of the second plate. In other words, the tip 66 of post protrudes out bore 50. Note that the fastener 70 may be threaded into hole 74 at this point in the procedure, but should not be fully tightened so as to allow for movement of the second plate 40 along post 60 (at least parallel to post axis 62, and possibly with additional degrees of freedom). The surgical tool 80 is then moved into engagement with the post 60 by inserting post distal tip 66 into base unit 90 and bringing drive gear 88 into engagement with gear teeth 67. The driver 81 is then rotated in a first direction (e.g., clockwise) to bring the base unit 90 into abutment with second plate 40. The driver 81 may be rotated by attaching a suitable drive unit, such as a power drive unit or a conventional ratchet drive, to the proximal section 84 of shaft 82 and then causing driver 81 to rotate about its longitudinal axis. Note that the ratcheting mechanism 92 helps prevent the surgical tool 80 from backing off of post 60 if the surgeon inadvertently attempts to rotate driver 81 in the wrong direction. Further rotation of the drive gear 88 forces the second plate 40 toward the first plate 20 along post 60. In particular, the inter-engagement of drive gear 88 and gear teeth 67 causes base unit 90 to press against second plate 40 as drive gear 88 is rotated. When the second plate 40 is moved sufficiently close to the first plate 20, such as when the plates 20,40 are clamped in the desired location on the spinous processes SP1,SP2, with teeth 28,48 biting into the opposing lateral sides of spinous processes SP1,SP2, the fastener 70 is then tightened by advancing fastener 70 to clamp against post 60 to lock the second plate 40 in position along post 60. Note that the notch 98 in base unit 90 allows access to fastener 70 for this locking. Once the second plate 40 is locked in position, the pawl 93 may be pivoted out of engagement with the drive gear 88, and driver 81 rotated in a second direction (e.g., counter-clockwise) to move the tool 80 off of post 60. The tool 80 is then removed from the surgical site. The surgical procedure then proceeds in the appropriate fashion, and the surgical site is closed.

Clamping plates 20,40 to the spinous processes SP1,SP2 helps maintain the alignment and spacing of the spinous processes SP1,SP2 while also providing resistance to spinal extension and flexion. Thus, engagement of plates 20,40 to the spinous processes SP1,SP2 resists movement of the spinous processes SP1,SP2 toward and away from one another as a result of spinal extension and flexion, respectively, or as a result of any other movement or condition. As can be appreciated, the second plate 40 is advantageously positionable along post 60 at an infinite number of positions, as the second plate 40 can conceptually slide to any number of positions along post 60 and be locked in the selected position by fastener 70. Thus, the implant 10 is able to accommodate a wide variety of patient morphologies.

While the above description has been in the context of an in-situ assembly of the first and second plates 20,40, in some embodiments the device 10 may be inserted in an already-assembled condition, with the second plate 40 already disposed on the post 60, and fastener 70 optionally engaged with second plate 40. Thus, the implant 10 may, in some embodiments, be assembled by the manufacturer or by medical personnel, prior to insertion of the implant 10 into the patient.

As will be appreciated, the first plate 20, second plate 40, post 60, fastener 70, and surgical tool 80 may each be made from any suitable biocompatible rigid materials such as titanium and its alloys, stainless steel, cobalt chrome, ceramics, relatively rigid polymers like carbon reinforced polyetheretherketone (PEEK), or the like, known in the art. As can be appreciated, the first plate 20, second plate 40, fastener 70, and tool 80 are advantageously distinct (i.e., separate) pieces from each other that are joined together during assembly.

In some embodiments, a sleeve (not shown) may be disposed on post 60 to provide additional support of the vertebrae to maintain or provide post-operative distraction between the spinous processes SP1,SP2. The sleeve may be osteoconductive if desired. For more information on sleeves, see U.S. Pat. No. 7,727,233.

While FIG. 1 shows an implant applied to vertebra L-4 and L-5, the implant 10 can be implanted on spinous processes at other levels. For example, levels up to T-3 may be appropriate sites. Also, plates 20,40 bridging more than one level may also be considered, optionally with multiple posts 60 disposed at suitable intervals.

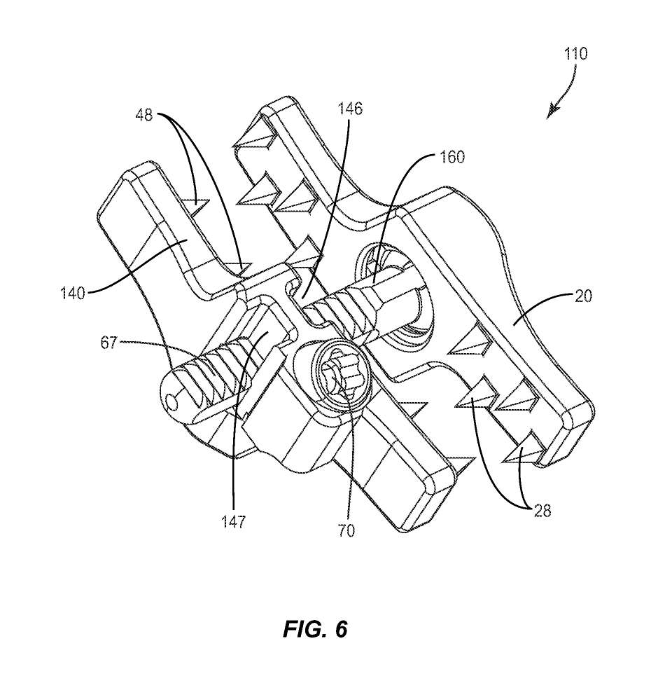

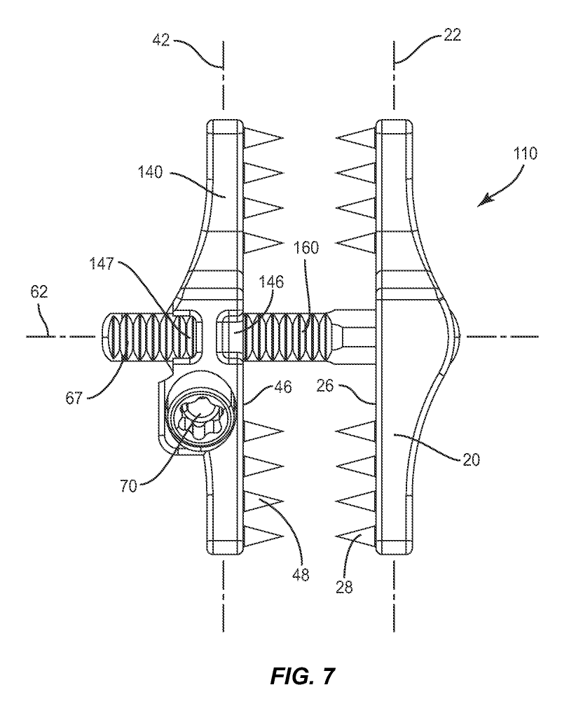

In the implant 10 illustrated above, the gear teeth 67 on post 60 are disposed to that the tips of gear teeth 67 point in a direction that is generally normal to both longitudinal axis 22 and post axis 62, and tool 80 is brought into engagement generally along post axis 62. However, such is not required in all embodiments. Indeed, in other embodiments, such as implant 110 of FIGS. 6-8, the gear teeth 67 on post 160 may point in a direction that is generally parallel to the longitudinal axis 22 and normal to post axis 62. For example, the post 160 shown in FIG. 6 has gear teeth 67 disposed so that the tips thereof point in a direction that is generally parallel to the longitudinal axis 22 and normal to post axis 62. Thus, the gear teeth 67 of FIG. 6 are conceptually rotated relative to post axis 62 approximately 90° from those in FIG. 2. Accordingly, the position and orientation of fastener hole 74 is modified in corresponding second plate 140, so that fastener 70 advantageously tightens against post 160 other than directly down onto gear teeth 67. For such an embodiment, surgical tool 180 may be used to gearingly drive the second plate 140 toward the first plate 20 via engagement with gear teeth 67, and this tool 180 may be brought into engagement generally normal to post axis 62.

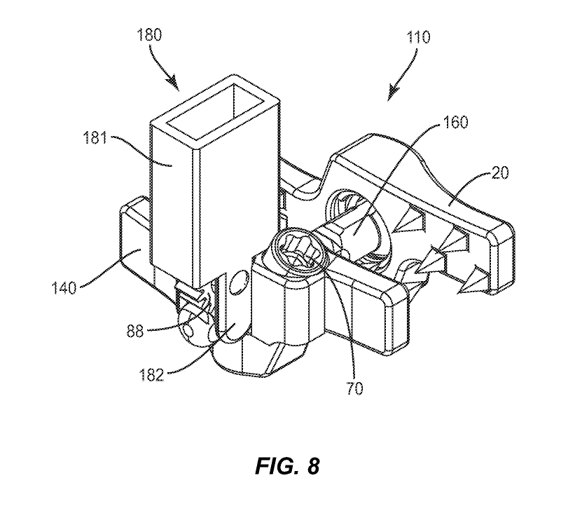

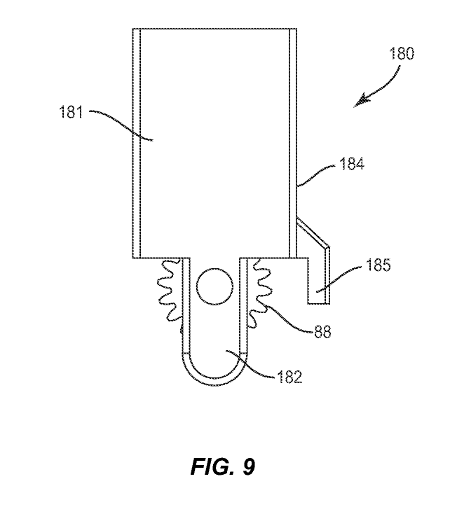

Surgical tool 180 of FIGS. 8-9 includes a housing 181 having two downwardly extending flanges 182 between which drive gear 88 is rotatably disposed. The housing 181 may have a generally rectangular cross-section with a generally flat forward face 184. A retention flange 185 may extend forwardly and downwardly (distally for the tool 180) from forward face 184. In order to facilitate operation of tool 180 with implant 110, second plate 140 advantageously includes a notch 147 on lateral face 47 for receiving flanges 182, and a notch 146 on medial face 46 for receiving retention flange 185.

The implantation procedure for implant 110 is similar to the implantation procedure of implant 10, with suitable modifications to accommodate the different configurations of post 160, second plate 140, and surgical tool 180. After the second plate 140 is slid toward the first plate 20, the post tip 66 becomes positioned distally beyond the lateral face 47 of second plate 140. In other words, the tip 66 of post 160 protrudes out bore 50. Note again that the fastener 70 may be threaded into hole 74 at this point in the procedure, but should not be fully tightened so as to allow for movement of the second plate 140 along post 160 (at least parallel to post axis 62, and possibly with additional degrees of freedom). The surgical tool 180 is then moved into engagement with the post 160 by moving tool 180 down into contact with post 160 and second wing 140 along a direction generally normal to the post axis 62 and longitudinal axis 42 (which corresponds to a posterior to anterior direction for most surgical situations). The drive gear 88 engages gear teeth 67 and flanges 182 abut lateral face 47 of second plate 140 in notch 147. Note that notch 147 advantageously is configured to provide abutment faces for flanges 182 to bear against, and also to provide space for drive gear 88 to rotate without contacting second plate 140; thus, notch 147 may have multiple “depths” in the direction toward first plate 20. In addition, retention flange 185 advantageously fits into notch 146, but advantageously does not extend farther toward second plate 20 than medial face 46. Drive gear 88 is then rotated in a first direction (e.g., clockwise), such as by attaching a suitable drive unit (e.g., a power drive unit or a manual drive) to the proximal end thereof. Note that the drive forces may be transmitted to drive gear 88 via a gear train (not shown) internal to tool 180. Rotation of drive gear 88 forces the second plate 140 toward the first plate 20 along post 160. In particular, the inter-engagement of drive gear 88 and gear teeth 67 causes housing 181 (typically flanges 182) to press against second plate 140 as drive gear 88 is rotated. When the second plate 140 is moved sufficiently close to the first plate 20, such as when the plates 20,140 are clamped in the desired location on the spinous processes SP1,SP2, with teeth 28,48 biting into the opposing lateral sides of spinous processes SP1,SP2, the fastener 70 is then tightened by advancing fastener 70 to clamp against post 160 to lock the second plate 140 in position along post 160. Note that the recessed nature of retention flange 185 helps prevent retention flange 185 from bearing against spinous processes SP1,SP2. Once the second plate 140 is locked in position, the tool 180 may be removed from the surgical site. The surgical procedure then proceeds in the appropriate fashion, and the surgical site is closed. It should be noted that if it is desired to move second plate 140 away from plate 20, rotation of drive gear 88 in a second direction (e.g., counter-clockwise) allows retention flange 185 to in effect pull second plate 140 away from first plate 20.

Also, any discussion above of implant 110, including plate 40 and post 60, and tool 80 with respect to assembly, materials, and alternative configurations also applies to implant 110, including plate 140 and post 160, and tool 180.

Note that as terms are used herein, conventional threading does not form “gear teeth” and threading a nut or the like on a threaded shaft is not “gearingly engaging” the nut (or the like) with the threaded shaft. Instead, the gear teeth 67 are configured so that the drive gears 88 of the present invention travels along the post 60,160 by rotation about an axis transverse to (advantageously orthogonal to) the post axis 62 rather than by rotating about the post axis 62.

The implant 10,110 may be used during surgical procedures on living patients. The implant may also be used in a non-living situation, such as within a cadaver, model, and the like. The non-living situation may be for one or more of testing, training, and demonstration purposes.

All U.S. patents, patent application publications, and applications mentioned above are hereby incorporated herein by reference in their entirety.

The present invention may, of course, be carried out in other ways than those specifically set forth herein without departing from essential characteristics of the invention. The present embodiments are to be considered in all respects as illustrative and not restrictive, and all changes coming within the meaning and equivalency range of the appended claims are intended to be embraced therein.

What is claimed is:

1. A method of attaching an implant to spinous processes comprising:
positioning a first plate on a first lateral side of the spinous processes;
positioning a second plate on a second lateral side of the spinous processes in spaced relation to the first plate;
positioning a post that extends outward from the first plate through an interspinous space formed between the spinous processes and through a bore in the second plate; the post extending along a post longitudinal axis from a proximal section attached to the first plate to a distal section positioned distally from the first plate; the post distal section having a plurality of external gear teeth thereon disposed in series at increasing spacing from the first plate;
removably coupling a tool to the gear teeth;
thereafter, moving the second plate along the post axis toward the first plate by engaging successive gear teeth of the post with the tool so that the tool pushes the second plate toward the first plate;
thereafter, locking the second plate relative to the post.

2. The method of claim 1:
wherein the first plate has a longitudinal axis;
wherein the gear teeth point in a direction generally parallel to the first plate longitudinal axis and generally normal to the post axis;
wherein engaging the gear teeth with the tool comprises rotating a drive gear of the tool about an axis that is generally normal to the first plate longitudinal axis and generally normal to the post axis.

3. The method of claim 1:
wherein the first plate has a longitudinal axis;
wherein the gear teeth point in a direction generally normal to the first plate longitudinal axis and generally normal to the post axis;
wherein engaging the gear teeth with the tool comprises rotating a drive gear of the tool, while engaged with the gear teeth, about an axis that is generally parallel to the first plate longitudinal axis and generally normal to the post axis.

4. The method of claim 1 further comprising thereafter decoupling the tool from the gear teeth.

5. The method of claim 1 further comprising engaging a fastener with the second plate prior to the locking; wherein the locking the second plate comprises advancing the fastener toward the post axis.

6. The method of claim 1 further comprising pivoting the post relative to the first plate prior to said locking.

7. The method of claim 1 wherein moving the second plate toward the first plate comprises causing protrusions on the first and second plates to bite into opposing lateral sides of adjacent spinous processes.

8. The method of claim 1 wherein the tool comprises a ratcheting mechanism; and wherein engaging successive gear teeth of the post with the tool comprises rotating a drive gear of the tool in a first direction and inhibiting rotation of the drive gear in a second opposite direction via the ratcheting mechanism.

9. The method of claim 1 wherein removably coupling the tool the gear teeth comprises:
after said positioning the first plate, said positioning the second plate, and said positioning the post, moving the tool in an anterior direction and into engagement with the implant;
mating a drive gear of the tool with one or more of the gear teeth of the post.

10. The method of claim 1 wherein engaging successive gear teeth of the post with the tool comprises rotating a drive gear of the tool in a first direction about a gear axis disposed normal to the post axis while the drive gear is in contact with the gear teeth of the post.

11. A spinal implant for attaching to adjacent spinous processes comprising:
a first plate sized to extend along a first lateral side of the spinous processes;
a second plate sized to extend along a second lateral side of the spinous processes, the second plate including a first bore that extends through the second plate between a medial surface that faces towards the first plate and an opposing outer surface;
an elongated post that extends along a post longitudinal axis from a proximal section attached to the first plate to a distal section positioned distally from the first plate; the distal section having a plurality of external gear teeth thereon disposed in series at increasing spacing from the first plate;
the first bore sized relative to the post for the second plate to be infinitely movable along the post and selectively locked in position therealong;
a fastener engageable with both the second wing and the post to selectively lock the second wing relative to the post.

12. The spinal implant of claim 11 wherein the second plate further comprises a fastener bore intersecting the first bore; wherein the fastener is disposed in the bore.

13. The spinal implant of claim 11 wherein the fastener is a setscrew.

14. The spinal implant of claim 11 wherein the gear teeth are disposed in series aligned normal to the post axis.

15. The spinal implant of claim 11 wherein the fastener engages the post from a direction orthogonal to the post axis.

16. The spinal implant of claim 11 wherein the first and second plates each comprise a plurality of protrusions extending toward each other; wherein the protrusions are configured to bite into the spinous processes.

17. The spinal implant of claim 11 wherein the gear teeth extend not more than partially circumferentially around the post axis.

18. The spinal implant of claim 11 wherein at least some of the gear teeth are disposed in the first bore.

19. The spinal implant of claim 11 wherein the first plate has a longitudinal axis; wherein the gear teeth point in a direction generally parallel to the first plate longitudinal axis and generally normal to the post axis.

20. The spinal implant of claim 11 wherein the first plate has a longitudinal axis; wherein the gear teeth point in a direction generally normal to the first plate longitudinal axis and generally normal to the post axis.
